(19) United States
(12) Patent Application Publication
(10) Pub. No.: US 2019/0192051 A1
(43) Pub. Date: Jun. 27, 2019

(54) DISPOSABLE SENSOR FOR NEUROMUSCULAR TRANSMISSION MEASUREMENT

(71) Applicant: General Electric Company, Schenectady, NY (US)

(72) Inventor: Outi Kristiina Savinen, Espoo (FI)

(21) Appl. No.: 15/853,610

(22) Filed: Dec. 22, 2017

(51) Int. Cl. A61B 5/11; A61B 5/0492; A61B 5/00

(52) U.S. Cl. CPC A61B 5/1106; A61B 5/0492; A61B 2562/164; A61B 5/6829; A61B 2562/0219; A61B 5/6825

(57) ABSTRACT

Sensors are provided for monitoring neuromuscular blockade in patients. In one embodiment, a sensor includes a first flexible substrate including a sensing element for measuring patient response to a stimulus, a second flexible substrate for providing the stimulus, the second flexible substrate including at least two electrodes, the first flexible substrate not connected to the second flexible substrate, and a common connector coupled to both the first flexible substrate and the second flexible substrate, the connector configured to couple to an NMT monitoring device.

### FIELD

Embodiments of the subject matter disclosed herein relate to medical devices, and more particularly, to monitoring neuromuscular transmission during a surgical procedure.

### BACKGROUND

Neuro Muscular Transmission (NMT) is the transfer of an impulse between a nerve and a muscle in the neuromuscular junction. NMT may be blocked in a patient undergoing a surgical procedure, for example, by neuromuscular blocking agents/drugs, which may cause transient muscle paralysis and prevent the patient from moving and breathing spontaneously. Thus, the level of neuromuscular block may be monitored to ensure appropriate block is provided for the given procedure.

### BRIEF DESCRIPTION

In one embodiment, a sensor includes a first flexible substrate including a sensing element for measuring patient response to a stimulus, a second flexible substrate for providing the stimulus, the second flexible substrate including at least two electrodes, the first flexible substrate not connected to the second flexible substrate, and a common connector coupled to both the first flexible substrate and the second flexible substrate, the connector configured to couple to an NMT monitoring device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example neuromuscular transmission (NMT) monitoring system including a first embodiment of an NMT sensor configuration.

FIG. 2 shows a second embodiment of an NMT sensor configuration.

FIG. 3 shows a third embodiment of an NMT sensor configuration.

FIG. 4 shows a fourth embodiment of an NMT sensor configuration.

FIG. 5 shows a fifth embodiment of an NMT sensor configuration.

FIG. 6 shows an example stimulating structure with printed leads.

FIGS. 7-8 show example stimulating structures with wire leads.

FIG. 9 shows an example kit of NMT sensors.

### DETAILED DESCRIPTION

The following description relates to various embodiments of a neuromuscular transmission (NMT) monitoring system configured to monitor an amount of neuromuscular blockage after the administration of muscle relaxants in patients during surgery. NMT is the transfer of an impulse between a nerve and a muscle in the neuromuscular junction. NMT may be blocked by neuromuscular blocking agents/drugs, which may cause transient muscle paralysis and prevent the patient from moving and breathing spontaneously. Additionally, muscle relaxation may be used during general anesthesia to enable endotracheal intubation and to provide the surgeon with optimal working conditions. At the end of a surgical procedure, the neuromuscular block is reversed such that neuromuscular activity may be returned to normal and that the patient may be able to breathe unassisted, before the removal of the endotracheal intubation (i.e. extubation). Thus, appropriate assessment of the degree of NMT block may be used for ensuring proper timing of intubation and for guiding intraoperative administration of neuromuscular blocking agents, maintaining a desired degree of intraoperative neuromuscular block, and ultimately preventing the occurrence of residual muscle paralysis.

An NMT monitor may be used to monitor muscle response to electrical stimulation of a motor nerve (e.g., the ulnar nerve). For example, an electrical stimulus may be provided at the ulnar nerve near the wrist and the response of the adductor pollicis muscle near the thumb may be monitored. In clinical settings, a nerve stimulator is attached to a motor nerve of the patient and an electrical stimulation current is applied to the patient before induction of anesthesia. A baseline value for the muscle response is recorded by the NMT monitor and used to normalize the muscle response once the muscle relaxant is administered. The evoked muscle responses may then be monitored via several methods, including the measurement of electrical response of the muscle (electromyography (EMG)) and the measurement of the degree of distortion or bending in a piezoelectric film attached to the muscle (kinemyography (KMG)). KMG measures mechanical activity based on the distortion or bending of the piezoelectric polymer strip placed within the bending sensor between the thumb and forefinger. In EMG, multiple electrodes may be used to record the compound muscle potential stimulated by the stimulus generator.

Thus, in EMG-based NMT monitoring systems, multiple sensing elements as well as a stimulating element are placed on the hand or foot of a patient. Each sensing element and stimulating element may be placed on the patient individually, and then the leads coupling each sensing element and stimulating element to the NMT monitoring system are individually coupled to the respective sensing or stimulating element. Therefore, attaching the EMG sensing and stimulating elements to a patient may be time consuming. Further, the EMG configuration described above be difficult to disinfect. While the KMG-based NMT monitoring system described above may include fewer sensing elements, KMG-based NMT monitoring may not be as accurate as EMG-based NMT monitoring.

According to embodiments disclosed herein, an NMT sensor for use in an NMT monitoring system may include a sensing structure comprised of a sensing element coupled to a single flexible substrate. The flexible substrate may be shaped to correspond to the anatomy to which the substrate is to be attached (e.g., a hand). The sensing element may include two or more sensing electrodes, with the sensing electrodes coupled to the substrate at select positions such that the sensing electrodes will be coupled to target anatomy once the sensing structure is attached to a patient. In other examples, the sensing element may include a piezoelectric element, an accelerometer, or combinations thereof (e.g., sensing electrodes and a piezoelectric element or accelerometer). The NMT sensor may also include a stimulating structure comprised of stimulating electrodes coupled to a separate flexible substrate that is not attached to or a part of the sensing structure. Each of the sensing structure and stimulating structure may include a respective electric lead (e.g., cable) that join at a common connector configured to electrically couple (e.g., via a trunk cable) the sensing structure and stimulating structure to an NMT monitoring system. By separating the stimulating structure from the sensing structure, additional flexibility in the placement of the stimulating electrodes and sensing element may be provided and any forces acting on the trunk cable (such as due to patient movement or inadvertent movement of the trunk cable by a clinician) may be split between the two separate leads, thus lowering the likelihood the sensing structure or stimulating structure may be accidentally removed from the patient. Further still, the sensing structure and stimulating structure (including the two leads and the common connector) may be disposable, thus eliminating the need to disinfect any of the components of the NMT sensor after patient use and reducing the risk of cross-contamination among patients.

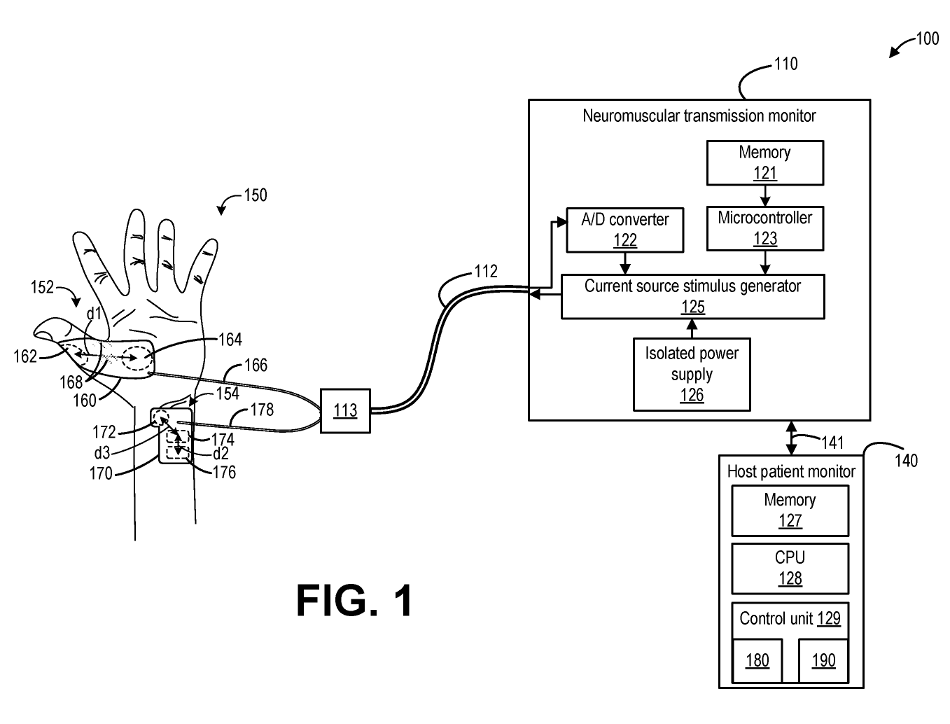

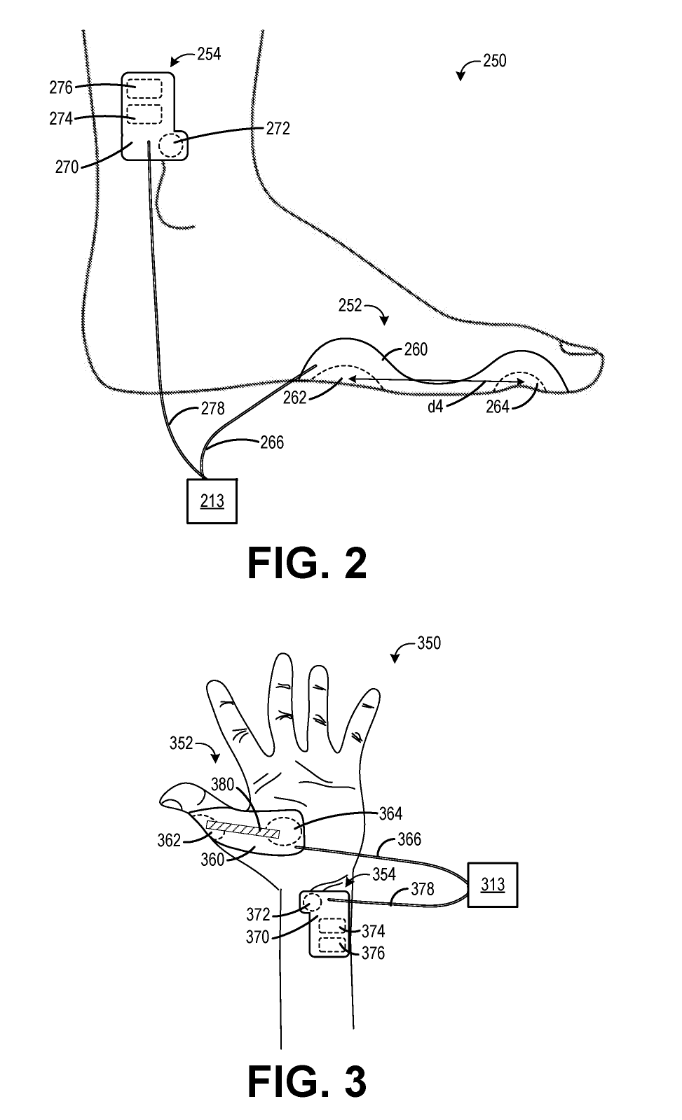

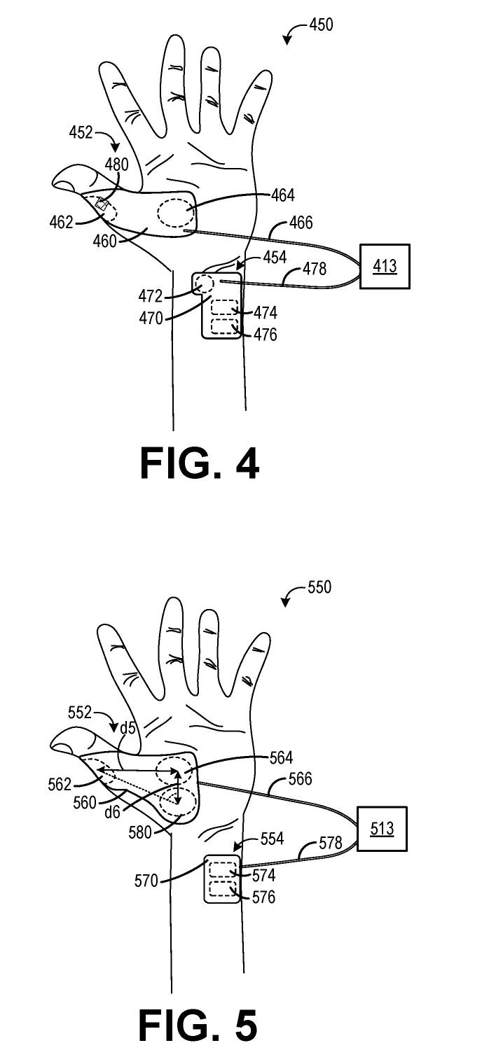

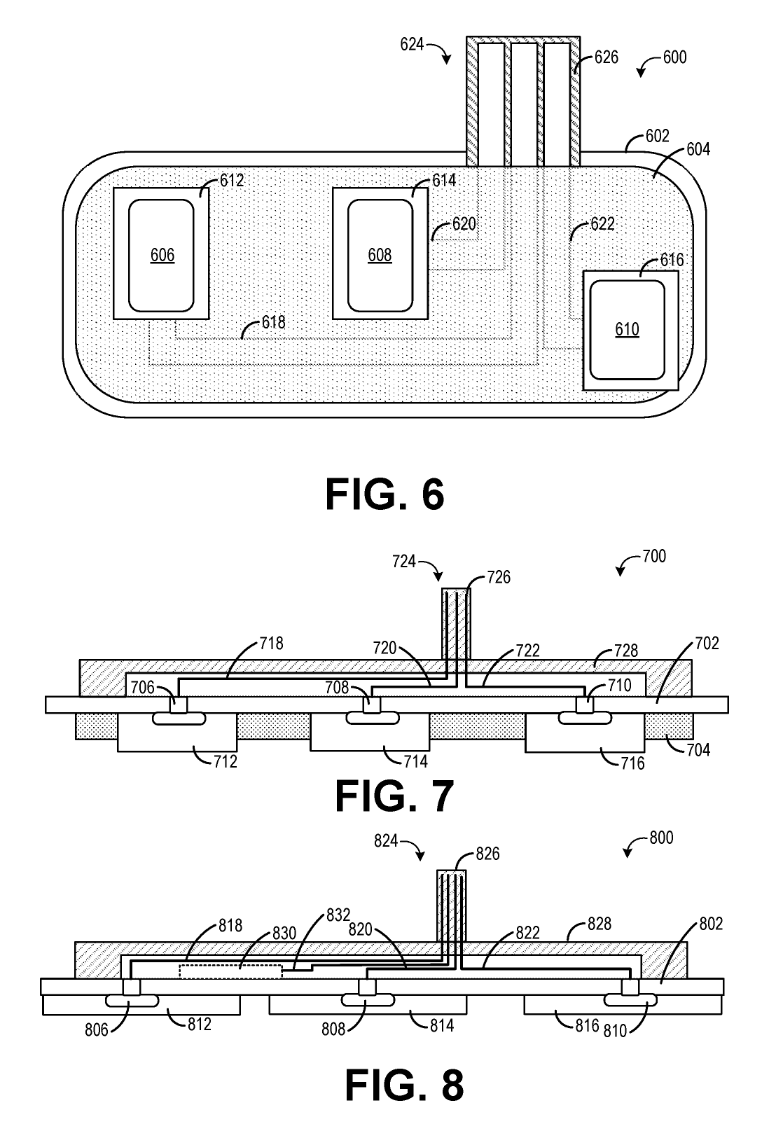

An example of a neuromuscular transmission monitoring system is provided in FIG. 1. The NMT monitoring system may include an NMT sensor adapted to be attached to a hand of a patient and comprising a stimulating structure configured to stimulate one or more nerves of a patient and a sensing structure that may include one or more electro-sensors which detect electrical activity of a muscle (referred to as an EMG sensor), where the electro-sensors are coupled to a single flexible substrate. The NMT monitoring system of FIG. 1 also includes a computing system including instructions to monitor neuromuscular block in patients in response to output from the NMT sensor. FIGS. 2-5 show various embodiments for the NMT sensor, including an NMT sensor with electro-sensors adapted to be placed on a foot of a patient (FIG. 2), an NMT sensor with a piezoelectric mechano-sensor as well as electro-sensors and adapted to be placed on a hand of a patient (FIG. 3), an NMT sensor adapted to be placed on a hand of a patient and including electro-sensors and an accelerometer (FIG. 4), and an NMT sensor adapted to be placed on a hand of a patient and including electro-sensors, with a grounding electrode present on the same flexible substrate as the electro-sensors (FIG. 5), rather than the grounding electrode being present on the same flexible substrate as the stimulating electrodes (as shown in FIGS. 1-4). The flexible substrates with sensing elements attached thereto may be manufactured according to a suitable configuration, including with printed leads (as shown by FIG. 6) or with lead wires (as shown in FIGS. 7 and 8).

FIG. 1 illustrates an example neuromuscular transmission (NMT) monitoring system 100 that is configured to monitor neuromuscular activity via an EMG-based NMT sensor. However, components of NMT monitoring system 100 may also be used to monitor neuromuscular activity via a KMG-based NMT sensor. NMT monitoring system 100 includes a neuromuscular transmission monitor 110 which is communicatively coupled to a host patient monitor 140 via a communication link 141. The neuromuscular transmission monitor 110 is configured to be coupled to a stimulating structure 154 (described in more detail below), for providing stimulation output (e.g., electrical stimuli) of varying type and frequency to the patient. The NMT monitor 110 also includes at least one input configured to couple to a sensing structure 152 that includes one or more elements for monitoring the evoked muscle response in response to the electrical stimuli provided by the neurostimulators. The signals detected by the transducers may then be converted into electrical signals by the A/D converter 122 of neuromuscular transmission monitor 110.

The amount of electrical stimulation provided to the stimulating structure 154 is controlled by a current stimulus generator 125 which receives command signals from microcontroller 123. Microcontroller 123 is linked to the user interface of control unit 129 of host patient monitor 140, which comprises of a display unit 190 and buttons/knobs 180. The type and frequency of the stimulation output may be adjusted manually by the user (manual mode) or be automatically chosen by the system (automatic mode). In one example, the type and frequency of the stimulation output may be adjusted by the user via pressing buttons or knobs 180 on the patient host monitor 140.

A power supply (not shown) may supply electricity to an isolated power supply 126 which in turn provides power to current source stimulus generator 125. The microcontroller 123 may be connected to the current source stimulus generator 125 to adjust the amount of electric current provided to the stimulating structure 154. The current stimulus generator 125 may generate different types of neurostimulation including train-of-four (TOF), single twitch (ST), double burst (DBS), post tetanic count (PTC), current range (e.g., 1-70 mA with 1 mA steps), pulse width/frequency (e.g., 100, 200, 300 μs, or 1 Hz, 2 Hz, etc.). Further, the types of neurostimulation may be chosen via a manual or an automatic stimulating mode. If a manual stimulating mode is chosen, then the user may input the desired neuromuscular stimulating types, current range, and pulse width and/or frequency via pressing button 180 of the host patient monitor 140, for example. Alternatively, if a touch-screen is used as the display unit (e.g., display unit 190 of host patient monitor 140), then user input may be provided via touch input to the touch-screen on the display unit.

If an automatic neurostimulation mode is chosen, microcontroller 123 of neuromuscular transmission monitor 110 may select a first neurostimulation type as its default setting, such as TOF stimulation, and based on the muscle response signals received from the sensing structure 152, the microcontroller may determine the optimal neurostimulation type, and report the muscle response signals to the user by displaying graphs and numbers (e.g., via display unit 190 of host patient monitor 140). The display unit 190 may display the muscle response data/information to the user and may also include alarm signals/message for alerting the user of potential sensor error.

Additionally, neuromuscular transmission monitor 110 may be connected to a host patient monitor 140 through a communication link 141. Host patient monitor 140 may include memory 127, CPU 128, and control unit 129. Memory 127 may have similar functions as memory 121. Control unit 129 may include control buttons/knobs 180 and display unit 190. The control buttons and knobs of control unit 129 may be configured to allow for user input. The display unit 190 may be configured to receive touch input from a user.

NMT sensor 150 includes a sensing structure 152 and a stimulating structure 154. Sensing structure 152 includes a flexible substrate 160 to which a sensing element is coupled. In the example depicted in FIG. 1, the sensing element includes two electrodes, a first electrode 162 and a second electrode 164. Flexible substrate 160 may be comprised of a suitable flexible, non-conductive material such as silicon, polyethylene terephthalate (PET), polyethylene (PE), or other thermoplastic resins. Flexible substrate 160 may be shaped to conform to the underlying anatomy (e.g., hand) and thus include a first region configured to be placed on top of a suitable region of the thumb, such as the distal interphalangeal joint of the thumb, for example, and a second region configured to be placed on top of a suitable region of the palm, such as the adductor pollicis in the thenar eminence, for example, with a connecting region coupling the first and second regions. The connecting region may be shaped to allow movement of the thumb relative to the palm (e.g., the connecting region may be countered to match the contour of the hand where the thumb extends from the palm) and may bend to accommodate differences in patient size, anatomy, and movement. In some examples, the connecting region (and/or other areas of the flexible substrate) may include areas of additional flexibility, such as areas 168. These areas may be comprised of a different material than the rest of the flexible substrate, where the different material is more flexible than the rest of the flexible substrate. The areas may accommodate flexing, bending, etc., of the flexible substrate in areas expected to be subject to movement. First electrode 162 may be positioned on the first region and second electrode 164 may be positioned on the second region. First electrode 162 and second electrode 164 may be spaced apart by a distance d1 (e.g., d1 may be the distance between centers of the electrodes). The spacing may be based on patient population average distances between target underlying anatomy (e.g., an average distance between the adductor pollicis in the thenar eminence and the distal interphalangeal joint of the thumb) or other suitable distance. By positioning the electrodes at these regions, consistent EMG signals may be obtained.

Sensing structure 152 is configured to output measurement of electrical activity sensed by electrodes 162 and 164 in response to nerve stimulation and when received at the neuromuscular transmission monitor is recorded as an EMG muscle response signal. Electrodes 162 and 164 are electro-sensors for measuring the action potential of muscle contraction in response to nerve stimulation. Sensing structure 152 includes a lead 166 configured to transmit electrical output from electrodes 162 and 164 to NMT monitor 110. Lead 166 may be comprised of insulated wires (e.g., copper wire) coupled to flexible substrate 160 at a first end and coupled to a common connector 113 at a second end. The connection between the electrodes and the lead is described in more detail below with respect to FIGS. 6-8. Connector 113 is configured to couple to NMT monitor 110, via trunk cable 112 in the illustrated example. Connector 113 is configured to be removeably attached to trunk cable 112, such that connector 113, lead 166, and flexible substrate 160 (with associated electrodes) may be detached from NMT monitor 110 after use. In some examples, connector 113 may couple directly to NMT monitor 110 (e.g., without an intervening cable) or connector 113 may include a communication module or other elements to wirelessly communicate with NMT monitor 110.

Stimulating structure 154 includes a flexible substrate 170, a grounding electrode 172, and two neurostimulating electrodes 174 and 176 which may apply an electrical stimulus to the patient's ulnar nerve at a pre-determined time interval. Electrodes 174 and 176 may be spaced apart by distance d2 and electrode 174 and grounding electrode 172 may be spaced apart by distance d3. As shown, distance d2 is smaller than distance d3. The distances d2 and d3 may be suitable distances of at least 0.5 cm. Flexible substrate 170 may be comprised of similar material as flexible substrate 160 and may be separate from flexible substrate 160, and thus may allow placement of stimulating structure 154 on a suitable anatomy, regardless of the size of the patient. As shown, the stimulating structure 154 may be placed directly on the wrist of the patient, where the electro-stimulation of the underlying nerve(s) may be strongest and least prone to attenuation by fat or other tissue. Stimulating structure 154 includes a lead 178 coupled to flexible substrate 170 at a first end and to connector 113 at a second end. Lead 178 may be comprised of insulated wires and may be configured to transmit electrical signals to electrodes 174 and 176 in order to stimulate the underlying nerve(s) during NMT monitoring. In this way, stimulating structure 154 may be detachable from MNT monitor 110 after use, along with sensing structure 152. Connector 113 may only be coupled to leads 166 and 178 (while being configured to couple to cable 112), and may not be coupled to other leads or inputs. In other examples where the grounding electrode is supported on a separate substrate (e.g., a substrate separate from the flexible substrates 160 and 170), connector 113 may be coupled to leads 166 and 178 as well as an additional lead coupled to the grounding electrode. In some examples, leads 166 and 178 may be fixedly coupled to connector 113.

Stimulating structure 154 and sensing structure 152 may have mechanisms for improving electrical contact to skin, such as conductive gel, and mechanisms for improving fixation to the skin, such as adhesives placed beneath the electrodes. Further, the electrodes may be suitable electrodes, such as silver/silver chloride electrodes. The electrodes may be disposable electrodes which can be discarded after a single use.

Flexible substrate 160 and flexible substrate 170 are not connected to one another other than the connection of each of the leads to the common connector. Thus, as used herein, the flexible substrates of the sensing structure and of the stimulating structure not being connected to each and/or being maintained separate may include each of the flexible substrates having a border that surrounds and defines each flexible substrate and the borders do not connect or overlap. Thus, a first border of the flexible substrate 160 is maintained continuously around the entirety of the flexible substrate 160, and a second border of the flexible substrate 170 is maintained continuously around the entirety of the flexible substrate 170. At least in some examples, the first border and the second border may not touch, overlap, or connect to each other when the NMT sensor is worn by a patient.

The type of neuromuscular stimulating outputs (e.g., output by stimulating structure 154) may include train-of-four (TOF), single twitch (ST), double burst (DBS) and post-tetanic count (PTC). In one example, TOF may typically use four brief (between 100 and 300 μs) current pulses (generally less than 70 mA) at 2 Hz, repeated every 10 to 20 s as electrostimulation. The resulting twitches (i.e. muscle response) may be measured and quantified for electromyographic response via the sensing structure 152. The first twitch (referred to as the T1 twitch) and the last twitch (referred to as the T4 twitch) are compared and the ratio of the last twitch to the first twitch may provide an estimate of the level of neuromuscular blockade (e.g., depth of anesthesia) experienced by the patient. The TOF ratio may range from 0 to 100%, for example. The electrical stimuli series may be spaced by ten or more seconds (generally 20 s is used to provide a margin of safety) to give a rest period for full restoration of steady state conditions, as faster stimulation results in smaller evoked responses. TOF is the most commonly used technique for monitoring the neuromuscular blockade in lightly-blocked patients as well in patients that are recovering from neuromuscular block. However, in deep muscle blockade condition (e.g., during deep sedation), the fourth twitch may be too weak to be detected and thus, may provide a TOF ratio of zero. In that case, the muscle response may be provided at TOF count (TC).

During NMT monitoring, information regarding the EMG muscle response signals received from sensing structure 152 may be sent to neuromuscular transmission monitor 110 via connector 113 and cable 112. In one example, muscle response signals from sensing structure 152 may be differentiated and further fed into a signal scaling and filtering circuit (not shown). After scaling the signal and filtering noise, the signal may be converted from an analog signal to a digital signal in analog-to-digital (A/D) converter 122 and sent to microcontroller 123 for processing. Further, the muscle response signals may also be amplified via an amplifier (not shown) before being transmitted into the A/D converter 122. The microcontroller 123, or processing unit, is connected to a memory 121 and once the signals are processed, the signal data may be displayed on the display unit 190 of the host patient monitor. In one example, the processed signals may be transmitted to the host patient monitor 140 and displayed on the display unit 190 in real-time. Further still, the processed signals may be updated and stored in memory 121. Memory 121 may be a conventional microcomputer which includes: a central processing unit (CPU), a read-only memory (ROM), a random access memory (RAM), and a conventional data bus.

Further still, the microcontroller may be configured to detect errors in the signal received from any of the sensing electrodes. The microcontroller may detect errors such as out-of-range values (e.g., negative values) and alert the user that the electrodes may not be placed properly (e.g., if the electrodes become loose or detached from the skin).

Control unit 129 may also include a user interface (not shown) which can be used to control operation of the NMT monitoring system 100, including controlling the input of patient data, changing the monitoring parameters (e.g. stimulus type, current range, frequency/pulse width, etc.), and the like. The user interface may also include a graphical user interface configured for display on a display device, such as display unit 190. The graphical user interface may include information to be output to a user (such as muscle response signals, patient data, etc.) and may also include menus or other elements through which a user may enter input the control unit 129.

Further, CPU 128 may process the input provided by the user and command a constant current source stimulus generator 125 to provide a stimulus waveform and current depending on the selected stimulus mode, current range, and pulse width/frequency. The neurostimulation type may be changed according to the patient's current and overall state of neuromuscular blockade. Further, a conversion module may be provided to convert muscle response value from a non-TOF stimuli into corresponding TOF data. For example, the conversion from PTC to TOF may be mapped using a linear or sigmoidal relationship model, and displayed as a TOF value on the display unit. In another example, the conversion module may further include a duration where a PTC evoked muscle responses may be measureable by TOF stimulation (e.g., if a PTC of 1 is measured, the conversion module may indicate that TOF may be measurable in 12 minutes).

Thus, the NMT sensor 150 described above provides for a single use, disposable sensor with separate sensing and stimulating parts (comprised of pre-attached electrode bodies that include electrodes coupled to a flexible substrate) intended for monitoring neuromuscular transmission (NMT). The pre-attached electrode bodies each have a supporting shape that may ease the sensor attachment procedure. The electrodes are released faster from the release liner than configurations where each electrode is provided separately, and the correct placement of the electrodes is easier to identify than with separate electrodes. The step where the user connects the correct lead to the specific electrode is eliminated, further easing attachment of the NMT sensor 150 to the patient. By configuring the NMT sensor 150 to be a single use sensor which is disposed after usage, the risk of cross-contamination between patients is minimized and the need for reprocessing of the sensor is eliminated. Further, the two separate flexible substrates provides for easier attachment of the NMT sensor to a patient's hand, which may also contain other medical devices (catheters, etc.) and a wrist band conveying patient identification information, relative to an NMT sensor having only a single body. Also attaching the NMT sensor in two parts makes the handling easier as the adhesive surfaces are less likely to touch unwanted surfaces (instead of skin).

The shape of sensing structure 152 and/or stimulating structure 154 shown in FIG. 1 is not limiting, as other shapes are possible without departing from the scope of this disclosure. The grounding electrode may be positioned in a suitable location, as long as the grounding electrode is not located directly on the nerve to be stimulated, nor on the same muscles that are measured. The electrodes (e.g., the sensing and/or stimulating electrodes) are positioned a suitable distance apart to prevent the electrodes from coming into electrical contact with each other, even while the patient is moving or is moved by the clinicians. The sufficient isolation between the electrodes may be ensured by having enough distance (>5 mm) between them; or by applying non-conducting material between the electrodes in the electrode element design (such as PE foam). The size of the needed conductive area of each electrode may depend on the used technology. For example, with high-conductive wet gel electrodes sufficient electrical contact (low impedance) with skin may be reached with smaller contact area than with solid gel with smaller amount of chloride ions. The stimulating electrodes may be sized to have some width in relation to the nerve location, to ease the application of the electrodes on the top of the nerve (e.g., make the nerve easier to stimulate). However, the conductive areas of the sensing electrodes may be sized to be smaller than the measured muscle, and the stimulating electrodes may be sized to be smaller than a given width (e.g., 5 cm), as the simultaneous stimulation of the adjacent nerves is to be avoided.

NMT sensor 150 is configured (e.g., shaped) to sense neuromuscular transmission at a hand of a patient. While such a configuration provides reliable and accurate NMT monitoring, in some circumstance (e.g., where a hand is not available), it may be desirable to monitor NMT on a different anatomy, such as a foot or face of a patient. FIG. 2 shows an embodiment of an NMT sensor 250 configured to be placed on a foot of a patient. NMT sensor 250 includes a sensing structure 252 and a stimulating structure 254. Sensing structure 252 includes a flexible substrate 260 to which a sensing element is coupled. In the example depicted in FIG. 2, the sensing element includes two electrodes, a first electrode 262 and a second electrode 264. Flexible substrate 260 may be similar to flexible substrate 160 (e.g., comprised of PET or other flexible material). Flexible substrate 260 may be shaped to conform to the underlying anatomy (e.g., foot) and thus include a first region configured to be placed on a suitable region of the foot, such as the arch of the foot, for example, and a second region configured to be placed on a suitable region of the foot, such as at the base of the big toe, for example, with a connecting region coupling the first and second regions. The connecting region may be shaped to allow movement of the toes relative to the rest of the foot (e.g., the connecting region may be contoured to match the contour of the foot) and may bend to accommodate differences in patient size, anatomy, and movement. First electrode 262 may be positioned on the first region and second electrode 264 may be positioned on the second region. By positioning the electrodes at these regions, consistent EMG signals may be obtained.

Sensing structure 252 is configured to output measurement of electrical activity sensed by electrodes 262 and 264 in response to nerve stimulation and when received at the neuromuscular transmission monitor is recorded as an EMG muscle response signal. Electrodes 262 and 264 are electro-sensors for measuring the action potential of muscle contraction in response to nerve stimulation. Electrodes 262 and 264 are spaced apart on the flexible substrate by a distance d4. In one example, distance d4 may be larger than distance d1 and may be based on the separation of the target underlying anatomy (e.g., the regions of the muscle expected to respond to the stimulation). Sensing structure 252 includes a lead 266 configured to transmit electrical output from electrodes 262 and 264 to an NMT monitor, such as NMT monitor 110. Lead 266 may be comprised of insulated wires (e.g., copper wire) coupled to flexible substrate 260 at a first end and coupled to a common connector 213 at a second end. The connection between the electrodes and the lead is described in more detail below with respect to FIGS. 6-8. Connector 213 is configured to couple to a trunk cable, such as cable 112, that is coupled to the NMT monitor. Connector 213 is configured to be removeably attached to the trunk cable, such that connector 213, lead 266, and flexible substrate 260 (with associated electrodes) may be detached from the NMT monitor after use.

Stimulating structure 254 includes a flexible substrate 270, a grounding electrode 272, and two neurostimulating electrodes 274 and 276 which may apply an electrical stimulus to the patient's nerve at a pre-determined time interval. Flexible substrate 270 may be separate from flexible substrate 260, and thus may allow placement of stimulation structure 254 on a suitable anatomy, regardless of the size of the patient. As shown, the stimulating structure 254 may be placed directly on the ankle of the patient, where the electro-stimulation of the underlying nerve(s) may be strongest and least prone to attenuation by fat or other tissue. Stimulating structure 254 includes a lead 278 coupled to flexible substrate 270 at a first end and to connector 213 at a second end. Lead 278 may be comprised of insulated wires and may be configured to transmit electrical signals to electrodes 274 and 276 in order to stimulate the underlying nerve(s) during NMT monitoring. In this way, stimulating structure 254 may be detachable from the NMT monitor after use, along with sensing structure 252.

Stimulating structure 254 and sensing structure 252 may have mechanisms for improving electrical contact to skin, such as conductive gel, and mechanisms for improving fixation to the skin, such as adhesives placed beneath the electrodes. Further, the electrodes may be suitable electrodes, such as silver/silver chloride electrodes. Further, the electrodes may be disposable electrodes which can be discarded after a single use. While FIGS. 1 and 2 show NMT sensors shaped to be placed on a hand and foot, respectively, it is to be understood that an NMT sensor as described herein may be placed in other locations, such as a face of a patient.

FIG. 3 illustrates an embodiment of an NMT sensor 350 that includes both electro-sensors and mechano-sensors. NMT sensor 350 includes a sensing structure 352 and a stimulating structure 354. Sensing structure 352 includes a flexible substrate 360 to which sensing elements are coupled. In the example depicted in FIG. 3, the sensing elements include two electrodes, a first electrode 362 and a second electrode 364, as well as a mechano-sensor 380. Flexible substrate 360 may be comprised of silicon, PET, PE, or other material and may be shaped similarly to flexible substrate 160, and hence description of flexible substrate 160 likewise applies to flexible substrate 360.

Sensing structure 352 is configured to output measurement of electrical activity sensed by electrodes 362 and 364 in response to nerve stimulation and when received at the neuromuscular transmission monitor is recorded as an EMG muscle response signal. Electrodes 362 and 364 are electro-sensors for measuring the action potential of muscle contraction in response to nerve stimulation.

Mechano-sensor 380 is configured for measuring muscle movement in response to nerve stimulation. Mechano-sensor 380 includes a mechano-sensing bending element. In the depicted example, the bending element is placed between the thumb and the forefinger. The bending element may comprise a piezoelectric polymer film which creates an electrical current in response to movement of any part of the polymer. When compressed or distorted, piezoelectric materials produce a charge proportional to the degree of alteration in shape. In FIG. 3, when the motor nerve is stimulated, thumb movement may cause a shape distortion in the bending element which in turn produces an electrical signal transmitted to an NMT monitor and recorded at the monitor as a KMG muscle response signal. Mechano-sensor 380 may be coupled to a top layer of flexible substrate 360, such that underlying electrodes 362 and/or 364 are in contact with the skin of the patient. In some examples, mechano-sensor 380 may be sandwiched intermediate two layers of the flexible substrate.

Sensing structure 352 includes a lead 366 configured to transmit electrical output from electrodes 162 and 164 and mechano-sensor 380 to an NMT monitor, such as NMT monitor 110. Lead 366 may be comprised of insulated wires (e.g., copper wire) coupled to flexible substrate 360 at a first end and coupled to a common connector 313 at a second end. The connection between the electrodes and the lead is described in more detail below with respect to FIGS. 6-8. Connector 313 is a non-limiting example of connector 113 and thus description of connector 113 likewise applies to connector 313. By including both EMG sensors and a KMG sensor, NMT sensor 350 may be configured to output both EMG and KMG muscle response signals. The NMT monitor may use both the EMG and KMG muscle response signals to monitor NMT, thereby allowing for filtering of noise from the electrical EMG signal based on the mechanical KMG signal, increasing robustness in the monitoring, and providing back-up/redundant sensing.

Stimulating structure 354 includes a flexible substrate 370, a grounding electrode 372, two neurostimulating electrodes 374 and 376 which may apply an electrical stimulus to the patient's ulnar nerve at a pre-determined time interval, and a lead 378. Stimulating structure 354 is a non-limiting example of stimulating structure 154 and thus description of stimulating structure 154 and all components comprising stimulating structure 154 likewise applies to stimulation structure 354.

FIG. 4 shows an embodiment of an NMT sensor 450 including a mechanical (e.g., movement) sensor in the form of an accelerometer. NMT sensor 450 includes a sensing structure 452 and a stimulating structure 454. Sensing structure 452 includes a flexible substrate 460 to which sensing elements are coupled. In the example depicted in FIG. 4, the sensing elements include two electrodes, a first electrode 462 and a second electrode 464, as well as accelerometer 480. Flexible substrate 460, electrodes 462 and 464, and lead 466 are non-limiting examples of flexible substrate 160, electrodes 162 and 164, and lead 166 and hence description of flexible substrate 160, electrodes 162 and 164, and lead 166 likewise apply to flexible substrate 360, electrodes 462 and 464, and lead 466. Accelerometer 480 is configured to measure movement (e.g., acceleration) of the thumb (or other underlying anatomy) that occurs responsive to electric stimulation. Accelerometer 480 may be a suitable accelerometer, such as a piezoelectric, piezoresistive, capacitive, or MEMS accelerometer. In FIG. 4, when the motor nerve is stimulated, thumb movement may cause accelerometer 480 to produce an electrical signal that is transmitted to an NMT monitor and recorded at the monitor as a KMG muscle response signal. Accelerometer 480 may be coupled to a top layer of flexible substrate 460, such that underlying electrode 462 is in contact with the skin of the patient. In some examples, accelerometer 480 may be sandwiched intermediate two layers of the flexible substrate.

Stimulating structure 454 includes a flexible substrate 470, a grounding electrode 472, two neurostimulating electrodes 474 and 476 which may apply an electrical stimulus to the patient's ulnar nerve at a pre-determined time interval, and a lead 478. Stimulation structure 454 is a non-limiting example of stimulation structure 154 and thus description of stimulation structure 154 and all components comprising stimulation structure 154 likewise applies to stimulation structure 454. Connector 413 is a non-limiting example of connector 113 and thus description of connector 113 likewise applies to connector 413. Further, while the NMT sensor 450 is configured for placement on a hand of a patient, in some examples a similar NMT sensor (e.g., that includes a sensing structure having two electrodes and an accelerometer) may be configured for placement on a foot or other anatomy of a patient.

FIG. 5 shows an embodiment of an NMT sensor 550 configured to measure EMG signals at a hand of a patient. NMT sensor 550 includes similar components as NMT sensor 150. However, in NMT sensor 550, the grounding electrode is positioned on the same flexible substrate as the sensing electrodes rather than on the flexible substrate of the stimulating structure. Thus, NMT sensor 550 includes a sensing structure 552 and a stimulating structure 554. Sensing structure 552 includes a flexible substrate 560 to which sensing elements are coupled. In the example depicted in FIG. 5, the sensing elements include two electrodes, a first electrode 562 and a second electrode 564. Flexible substrate 560, electrodes 562 and 564, and lead 566 are non-limiting examples of flexible substrate 160, electrodes 162 and 164, and lead 166 and hence description of flexible substrate 160, electrodes 162 and 164, and lead 166 likewise apply to flexible substrate 560, electrodes 562 and 564, and lead 566. Sensing structure 552 further includes a grounding electrode 580, which may be placed at a centerline over the flexor retinaculum at the palmar side of the wrist or other suitable location. To accommodate the grounding electrode 580, flexible substrate 560 may be longer and/or wider than flexible substrate 160, at least in the region where the grounding electrode is positioned. Electrodes 562 and 564 may be spaced apart by distance d5, which may be the same distance as d1 described above with respect to FIG. 1. Electrode 564 and grounding electrode 580 may be spaced apart by a distance d6, which may be smaller than d5 and may have a minimum distance of 0.5 cm. Electrodes 562 and 564 may be positioned on-axis with each other, and electrode 564 and grounding electrode 580 may also be positioned on-axis with each other along an axis that is perpendicular to the axis along which electrodes 562 and 564 are aligned. Thus, electrode 562 and grounding electrode 580 may be aligned along an axis that is angled relative to the axis along which electrode 564 and grounding electrode 580 are aligned, such as angle of 45° or 60°.

Stimulating structure 554 includes a flexible substrate 570, two neurostimulating electrodes 574 and 576 which may apply an electrical stimulus to the patient's ulnar nerve at a pre-determined time interval, and a lead 578. Stimulating structure 554 is a non-limiting example of stimulating structure 154 and thus description of stimulating structure 154 and the components comprising stimulating structure 154 likewise applies to stimulating structure 554. In some examples, flexible substrate 570 may be shorter and/or narrower than flexible substrate 170 due to the lack of a grounding electrode on flexible substrate 570. Connector 513 is a non-limiting example of connector 113 and thus description of connector 113 likewise applies to connector 513.

FIG. 6 schematically illustrates a top-down view of an example configuration for a stimulating structure 600 including a flexible substrate housing a plurality of electrodes. Simulating structure is a non-limiting example of stimulating structures 154, 254, 354, 454, and 554. While FIG. 6 is described with respect to a stimulating structure, the configuration described below likewise may be applied to a sensing structure. Stimulating structure 600 includes a first layer comprising a flexible substrate 602. Flexible substrate 602 may be comprised of silicon, polyethylene terephthalate (PET), PE, or other suitable flexible, non-conductive material. Flexible substrate 602 may be comprised of a single layer of material, or it may be comprised of more than one layer of material glued or otherwise coupled together. Flexible substrate 602 may be comprised of a continuous layer (or layers) of material (other than including cut-outs or openings to accommodate electrodes where appropriate), or flexible substrate 602 may be comprised of multiple segments of material joined together. A second layer of stimulating structure 600 includes a layer of adhesive foam 604. A plurality of electrodes may be coupled to flexible substrate 602 and/or adhesive foam 604, including a first electrode 606, a second electrode 608, and a third electrode 610 (in some examples, third electrode 610 may be omitted from stimulating structure 600). In some examples, the layer of adhesive foam 604 may include openings to accommodate each electrode, and each electrode may be directly coupled to the flexible substrate 602. In other examples, each electrode may be directly coupled to the layer of adhesive foam. In either example, front faces of the electrodes are not covered by the adhesive foam such that the electrodes are capable of making electrical contact with the skin of a patient.

Each electrode may be surrounded by a cushion of conductive gel in a sponge, including cushion 612, cushion 614, and cushion 616. The cushions of conductive gel may enhance electrical coupling between the electrodes and skin of the patient. The cushions may be coupled directly to the flexible substrate or to the layer of adhesive foam. While not shown in FIG. 6, in some examples, the conductive gel sponges may be coupled across the front faces of the electrodes, such that the electrodes electrically contact the skin of the patient via the conductive gel.

Each electrode is coupled to a lead. As shown, electrode 606 is coupled to lead 618, electrode 608 is coupled to lead 620, and electrode 610 is coupled to lead 622. Each lead may be comprised of conductive ink that is printed, painted, or otherwise applied to flexible substrate 602. As such, the leads may be sandwiched intermediate flexible substrate 602 and the layer of adhesive foam 604. The conductive ink may include carbon, silver, or other conductive particles suspended in ink. Each of the leads may be joined to form a collective lead 624 that couples stimulating structure 600 to a connector (such as connector 113). Collective lead 624 may include each of lead 618, lead 620, and lead 622 coupled to and/or encapsulated by a non-conductive material, such as non-conductive paint, rubber, or other material.

While not shown in FIG. 6, a sensing structure may be configured in a similar manner, including electrodes coupled to a flexible substrate and with leads comprised of conductive ink printed on the flexible substrate. In examples where a piezoelectric mechano-sensor or accelerometer is included in addition (or alternative) to the electrodes, the mechano-sensor or accelerometer may be coupled to the flexible substrate (e.g., on the opposite side as the electrodes) and may be coupled to a lead comprised of conductive ink.

Rather than including printed leads, some stimulating or sensing structures may include lead wires. FIGS. 7 and 8 show example configurations for sensing or stimulating structures comprising lead wires. FIG. 7 schematically illustrates a cross-sectional view of an example configuration for a stimulating structure 700 including a flexible substrate housing a plurality of electrodes. Stimulating structure 700 is a non-limiting example of stimulating structure 154, 254, 354, 454, and 554. While FIG. 7 is described with respect to a stimulating structure, the configuration described below likewise may be applied to a sensing structure. Stimulating structure 700 includes a first layer comprising a flexible substrate 702. Flexible substrate may be comprised of silicon, polyethylene terephthalate (PET), PE, or other suitable flexible, non-conductive material. Flexible substrate 702 may be comprised of a single layer of material, or it may be comprised of more than one layer of material glued or otherwise coupled together. Flexible substrate 702 may be comprised of a continuous layer (or layers) of material (other than including cut-outs or openings to accommodate electrodes where appropriate), or flexible substrate 702 may be comprised of multiple segments of material joined together. A second layer of stimulating structure 700 includes a layer of adhesive foam 704. A plurality of electrodes may be coupled to flexible substrate 702, including a first electrode 706, a second electrode 708, and an optional third electrode 710. In some examples, the layer of adhesive foam 704 may include openings to accommodate each electrode, and each electrode may be directly coupled to the flexible substrate 702.

Each electrode may be surrounded by a cushion of conductive gel in a sponge, including cushion 712, cushion 714, and cushion 716. The cushions of conductive gel may enhance electrical coupling between the electrodes and skin of the patient. The cushions may be coupled directly to the flexible substrate and/or to the layer of adhesive foam. As shown in FIG. 7, the conductive gel sponges may be coupled across the front faces of the electrodes, such that the electrodes electrically contact the skin of the patient via the conductive gel.

Each electrode is coupled to a lead. As shown, electrode 706 is coupled to lead 718, electrode 708 is coupled to lead 720, and electrode 710 is coupled to lead 722. Each lead may be comprised of conductive wire or wires, such as copper wire. Each of the leads may be bundled to form a collective lead 724 that couples stimulating structure 700 to a connector (such as connector 113). Collective lead 724 may include each of lead 718, lead 720, and lead 722 coupled to/encapsulated in a non-conductive material, such as rubber or other material. A layer of insulating material 728 may be coupled to flexible substrate 702, on an opposite of the electrodes, and may surround and/or encapsulate the conductive wire leads.

While not shown in FIG. 7, a sensing structure may be configured in a similar manner, including electrodes coupled to a flexible substrate and with leads comprised of conductive wire. In examples where a piezoelectric mechano-sensor or accelerometer is included in addition (or alternative) to the electrodes, the mechano-sensor or accelerometer may be coupled to the flexible substrate (e.g., on the opposite side as the electrodes) and may be coupled to a lead comprised of conductive wire.

FIG. 8 schematically illustrates a cross-sectional view of an example configuration for a stimulating structure 800 including a flexible substrate housing a plurality of electrodes. Stimulating structure 800 is a non-limiting example of stimulating structure 154, 254, 354, 454, and 554. While FIG. 8 is described with respect to a stimulating structure, the configuration described below likewise may be applied to a sensing structure. Stimulating structure 800 includes a first layer comprising a flexible substrate 802. Flexible substrate may be comprised of silicon, polyethylene terephthalate (PET), PE, or other suitable flexible, non-conductive material. Flexible substrate 802 may be comprised of a single layer of material, or it may be comprised of more than one layer of material glued or otherwise coupled together. Flexible substrate 802 may be comprised of a continuous layer (or layers) of material (other than including cut-outs or openings to accommodate electrodes where appropriate), or flexible substrate 802 may be comprised of multiple segments of material joined together. A plurality of electrodes may be coupled to flexible substrate 802, including a first electrode 806, a second electrode 808, and an optional third electrode 810.

Each electrode may be surrounded by a layer of adhesive conductive solid gel, including layer 812, layer 814, and layer 816. The layers of conductive gel may enhance electrical coupling between the electrodes and skin of the patient and maintain the structure coupled to the skin of the patient. The layers may be coupled directly to the flexible substrate. As shown in FIG. 8, the conductive gel layers may be coupled across the front faces of the electrodes, such that the electrodes electrically contact the skin of the patient via the conductive gel. The conductive solid gel is a relatively stable conductor, in that it may be less likely to run or exude from the electrode area than the conductive wet gel in the sponge described above, and may have a longer shelf life than the conductive wet gel. However, the conductive wet gel in the sponge may be a more effective conductor which quickly moisturizes the dry top surface of the skin (Stratum Corneum) and thus a good, low impedance electrical contact is reached in short time.

Each electrode is coupled to a lead. As shown, electrode 806 is coupled to lead 818, electrode 808 is coupled to lead 820, and electrode 810 is coupled to lead 822. Each lead may be comprised of conductive wire or wires, such as copper wire. Each of the leads may be bundled to form a collective lead 824 that couples stimulating structure 800 to a connector (such as connector 113). Collective lead 824 may include each of lead 818, lead 820, and lead 822 coupled to/encapsulated in a non-conductive material, such as rubber or other material. A layer of insulating material 828 may be coupled to flexible substrate 802, on an opposite of the electrodes, and may surround and/or encapsulate the conductive wire leads.

While not shown in FIG. 8, a sensing structure may be configured in a similar manner, including electrodes coupled to a flexible substrate and with leads comprised of conductive wire. In examples where a piezoelectric mechano-sensor or accelerometer is included in addition (or alternative) to the electrodes, the mechano-sensor or accelerometer may be coupled to the flexible substrate (e.g., on the opposite side as the electrodes) and may be coupled to a lead comprised of conductive wire. For example, FIG. 8 shows an accelerometer 830 coupled to flexible substrate 802, opposite the electrodes. Accelerometer 830 is coupled to a lead 832 (e.g., comprised of conductive wire), and lead 832 may be bundled with the other leads at the overall lead 824.

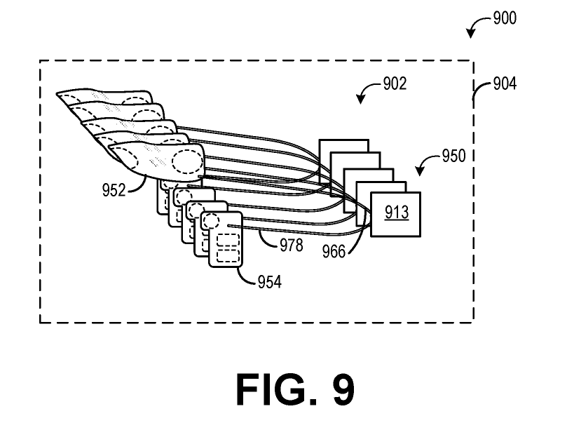

In some examples, a plurality of NMT sensors may be packaged together in a kit. FIG. 9 shows an example kit 900 including a plurality of NMT sensors 902. The plurality of NMT sensors 902 includes a first NMT sensor 950. NMT sensor 950 is a non-limiting example of NMT sensor 150 and thus may include the components of NMT sensor 150 described above with respect to FIG. 1, including a sensing structure 952, stimulating structure 954, leads 966 and 978, and a connector 913. The other NMT sensors of the plurality of NMT sensor 902 may be configured similarly to NMT sensor 950. The plurality of NMT sensors 902 may be packaged in a suitable packaging 904, such as a box.

The technical effect of an NMT sensor comprising a first flexible substrate with muscle response sensing element(s) attached thereon and a second flexible substrate with stimulating electrodes attached thereon is adaptivity to a desired anatomical monitoring site, enhanced ergonomics (including elimination of folded parts when attached to a patient of relatively small size, such as a child), and reduced pulling forces on the cables.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A sensor for monitoring neuromuscular transmission (NMT) of a patient, comprising:
a first flexible substrate including a sensing element for measuring patient response to a stimulus;
a second flexible substrate for providing the stimulus to the patient, the second flexible substrate including at least two electrodes, the first flexible substrate not connected to the second flexible substrate; and
a common connector coupled to both the first flexible substrate and the second flexible substrate, the connector configured to couple to an NMT monitoring device.

2. The sensor of claim 1, wherein the sensing element comprises at least two electrodes for measuring electrical activation of a muscle of the patient.

3. The sensor of claim 1, wherein the sensing element comprises a mechanical sensor for measuring mechanical activation of a muscle of the patient.

4. The sensor of claim 3, wherein the mechanical sensor comprises a piezoelectric bending element or an accelerometer.

5. The sensor of claim 1, further comprising a first flexible lead coupled to the first flexible substrate and to the connector and a second flexible lead coupled to the second flexible substrate and to the connector.

6. The sensor of claim 5, wherein the first flexible lead is configured to electrically couple the sensing element to the connector and the second flexible lead is configured to electrically couple the at least two electrodes to the connector.

7. The sensor of claim 6, wherein the first flexible lead and the second flexible lead each comprises one or more conductive wires encapsulated in a non-conductive material.

8. The sensor of claim 6, wherein the first flexible lead and the second flexible lead each comprises one or more strips of conductive ink printed on a non-conductive material.

9. The sensor of claim 1, wherein the sensing element comprises at least two electrodes for measuring electrical activation of a muscle of the patient and a piezoelectric bending element for measuring mechanical activation of the muscle of the patient.

10. The sensor of claim 1, wherein the sensing element comprises at least two electrodes for measuring electrical activation of a muscle of the patient and an accelerometer for measuring mechanical activation of the muscle of the patient.

11. The sensor of claim 1, wherein the first flexible substrate is shaped to conform to a hand of the patient.

12. The sensor of claim 1, wherein the first flexible substrate is shaped to conform to a foot of the patient.

13. The sensor of claim 1, wherein the first flexible substrate comprises a single, continuous layer of non-conductive material, and the second flexible substrate comprises a single, continuous layer of non-conductive material.

14. A sensor for monitoring neuromuscular transmission (NMT), comprising:
a first flexible substrate supporting at least two first electrodes for measuring muscle electrical response to a stimulus and a mechanical sensor for measuring muscle mechanical response to the stimulus;
a second flexible substrate supporting at least two second electrodes for providing the stimulus, the second flexible substrate not connected to the first flexible substrate; and
a common connector coupled to both the first flexible substrate and the second flexible substrate, the connector configured to couple to an NMT monitoring device.

15. The sensor of claim 14, wherein the at least two first electrodes are coupled to a first side of the first flexible substrate and the mechanical sensor is coupled to a second, opposite side of the first flexible substrate.

16. The sensor of claim 14, wherein the mechanical sensor comprises a piezoelectric bending element or an accelerometer.

17. The sensor of claim 14, further comprising a grounding electrode supported on the first flexible substrate or second flexible substrate.

18. A kit, comprising:
a plurality of neuromuscular transmission (NMT) sensors packaged in a common packaging, each NMT sensor of the plurality of NMT sensors comprising:
a first flexible substrate supporting a sensing element for measuring patient response to a stimulus and coupled to a first lead;
a second flexible substrate supporting at least two electrodes for providing the stimulus and coupled to a second lead, the second flexible substrate not connected to the first flexible substrate; and
a common connector coupled to both the first lead and the second lead, the connector configured to couple to an NMT monitoring device.

19. The kit of claim 18, wherein each sensing element comprises at least two electrodes.

20. The kit of claim 18, wherein the first lead and the second lead each comprise a plurality of conductive wires encapsulated in a flexible insulating material.
